(19) United States
(12) Patent Application Publication
(10) Pub. No.: US 2021/0016019 A1
(43) Pub. Date: Jan. 21, 2021

(54) FLOW-STOP ENABLED MOUTHPIECE, AND PORTABLE NEBULIZER THEREOF

(71) Applicant: Convexity Scientific Inc., Fairfield, CT (US)

(72) Inventors: Vincent F. Carrubba, Baldwin, NY (US); Ralph Finger, Westport, CT (US)

(21) Appl. No.: 16/931,745

(22) Filed: Jul. 17, 2020

Related U.S. Application Data
(60) Provisional application No. 62/876,377, filed on Jul. 19, 2019.

(51) Int. Cl. A61M 11/00

(52) U.S. Cl. CPC A61M 11/00

(57) ABSTRACT

The present disclosure discloses an aerosol inflow port, and an aerosol outflow port; wherein an aerosol passage is formed between the aerosol inflow port and the aerosol outflow port, such that in a normal spraying state, the aerosol enters the aerosol passage from the aerosol inflow port and is discharged from the aerosol outflow port, and an outflow stopper is provided on a bottom wall inner surface of the mouthpiece, and condensation of the aerosol is stopped by the outflow stopper when flowing towards the aerosol outflow port. Besides, the present disclosure further discloses a portable nebulizer adopting the mouthpiece. The present disclosure provides an advantage of effectively mitigating direct flow of the condensed aerosol into the mouth.

### CROSS REFERENCE TO RELATED APPLICATION

This application claims priority to and incorporates by reference U.S. provisional patent application No. 62/876,377 filed Jul. 19, 2019.

### FIELD

The present disclosure relates to the field of medical equipment, and more particularly relates to a flow-stop enabled mouthpiece, and a portable nebulizer.

### BACKGROUND

Nebulizers are very common medical equipment in modern medicine. Nebulizers are provided in many varieties, a most common one of which is a mask-type nebulizer used in hospitals. Portable nebulizers are also available.

### SUMMARY

Embodiments provide a mouthpiece of a nebulizer, which may effectively mitigate direct flow of condensation into the mouth.

In an embodiment, a flow-stop enabled mouthpiece comprises an aerosol inflow port and an aerosol outflow port; wherein an aerosol passage is formed between the aerosol inflow port and the aerosol outflow port, such that in a normal spraying state, the aerosol enters the aerosol passage from the aerosol inflow port and is discharged from the aerosol outflow port, characterized in that an outflow stopper is provided on a bottom wall inner surface of the mouthpiece, and condensation of the aerosol is stopped by the outflow stopper when flowing towards the aerosol outflow port.

Embodiments may provide the following advantages:

An aerosol passage is provided inside the mouthpiece. When a relatively large amount of aerosol is present in the mouthpiece, some small-sized condensation will be condensed into relatively large-sized condensation; when the nebulizer is shaken or moved to another location, the aerosol condensation in the mouthpiece will flow inside the mouthpiece; while if the mouthpiece tilts downwardly, the condensation will directly flow into the user's mouth. However, by providing the outflow stopper, the aerosol condensation will be stopped by the outflow stopper when flowing towards the aerosol outflow port, such that most of the condensation will remain in the mouthpiece rather than directly flowing into the user's mouth. Because the nebulizer is not activated for an extended prior of time for each, a user only needs to shake off the condensation or wipe it off upon the next use.

In an embodiment, the outflow stopper comprises an outflow stopping convex rib protruding from a bottom wall inner surface of the mouthpiece.

In an embodiment, a protrusion height of the outflow stopping convex rib ranges from 1 mm to 6 mm.

In an embodiment, the outflow stopper comprises an outflow stopping groove provided on a bottom wall inner surface of the mouthpiece.

In an embodiment, a depth of the outflow stopping groove ranges from 1 mm to 5 mm.

In an embodiment, the outflow stopper extends from a left wall inner surface of the mouthpiece to a right wall inner surface of the mouthpiece.

In an embodiment, an outer appearance of the aerosol outflow port is of a rectangular, round, or oval shape.

The present disclosure further discloses a portable nebulizer, comprising a housing, a nebulize unit provided to the housing, and a mouthpiece movably mounted on the housing, wherein the mouthpiece refers to the mouthpiece in any solution above, and in a normal spraying state, the aerosol inflow port is fitted to the nebulize unit.

In an embodiment, a receiving groove is provided on the housing, such that when the nebulizer is in a non-spraying state, the mouthpiece is received in the receiving groove, and when the nebulizer is in a spraying state, the mouthpiece is opened and has a working angle relative to the housing.

In an embodiment, the working angle ranges from 70° to 95°, and an opening of the aerosol inflow port extends from an end wall of the mouthpiece to a bottom wall outer surface of the mouthpiece.

These characteristics and advantages of the present disclosure will be described in detail through the illustrated embodiments and the accompanying drawings below.

### BRIEF DESCRIPTION OF THE DRAWINGS

Hereinafter, the present disclosure will be described in further detail with reference to the accompanying drawings:

FIG. 1 is a schematic diagram of a mouthpiece in Embodiment 1 of the present disclosure in a received state;

FIG. 2 is a schematic diagram of the mouthpiece in Embodiment 1 of the present disclosure in a normal spraying state;

FIG. 3 is a side schematic diagram of the mouthpiece in Embodiment 1 of the present disclosure in a normal spraying state;

FIG. 4 is a first structural schematic view of the mouthpiece in Embodiment 1 of the present disclosure;

FIG. 5 is a second structural schematic view of the mouthpiece in Embodiment 1 of the present disclosure;

FIG. 6 is an internal structural schematic view of the mouthpiece in Embodiment 1 of the present disclosure;

FIG. 7 is a backflow schematic view of condensation in Embodiment 1 of the present disclosure;

FIG. 8 is a structural schematic view of a bottom wall inner surface of the mouthpiece in Embodiment 1 of the present disclosure;

FIG. 9 is an internal air flow schematic view of the mouthpiece in Embodiment 1 of the present disclosure;

FIG. 10 is a structural schematic view of a stopper member in Embodiment 2 of the present disclosure;

FIG. 11 is a schematic view of a shape of an aerosol outflow port of the mouthpiece in Embodiment 3 of the present disclosure; and

FIG. 12 is a flowchart illustrating a method of operating a nebulizer.

### DETAILED DESCRIPTION

Hereinafter, the technical solutions of the embodiments of the present disclosure will be explained and illustrated with reference to the accompanying drawings corresponding to the embodiments of the present disclosure. Other embodiments obtained by those skilled in the art without exercise of inventive work based on the examples in the embodiments all fall within the protection scope of the present disclosure.

In the description below, the orientation or position relationships indicated by the terms “inner,” “outer,” “upper,” “lower,” “left,” and “right,” etc. are intended only for facilitating or simplifying description of the present disclosure, not for indicating or implying that the devices or elements have to possess those specific orientations and have to be configured and operated with those specific orientations; therefore, they should not be understood as limitations to the present disclosure.

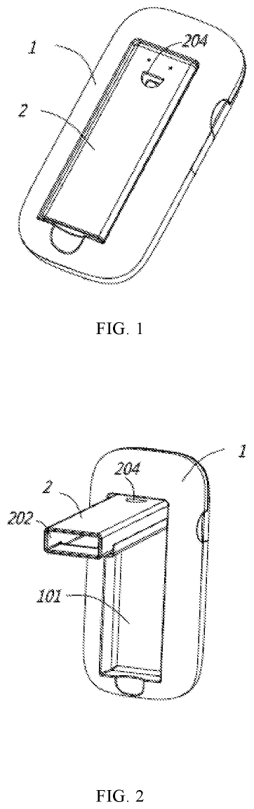

FIGS. 1-9 show an embodiment a mouthpiece 2. FIGS. 1-3 are schematic diagrams of the mouthpiece 2 mounted on a housing 1 of the nebulizer; the mouthpiece 2 according to this embodiment comprises an aerosol inflow port 201 and an aerosol outflow port 202; wherein an aerosol passage is formed between the aerosol inflow port 201 and the aerosol outflow port 202; a main air inlet 203 is further provided on the mouthpiece 2, the main air inlet 203 being in communication with the aerosol passage, such that in a normal spraying state, the aerosol produced by a nebulize unit on the nebulizer enters the aerosol passage from the aerosol inflow port 201 and is discharged from the aerosol outflow port 202, wherein a mouth of a user inhales from the aerosol outflow port 202 such that the aerosol is inhaled into the human body.

This embodiment also improves a forward flow direction of condensation in the mouthpiece 2. The forward flow direction here refers to a direction from the aerosol inflow port 201 towards the aerosol outflow port 202. An aerosol passage is provided inside the mouthpiece 2. When a relatively large amount of aerosol is present in the mouthpiece 2, some small-sized condensation will be condensed into relatively large-sized condensation; the mouthpiece 2 tends to tilt towards the user from time to time when in normal use, such that the condensation also tends to flow towards the aerosol outflow port 202; however, because the aerosol outflow port 202 is directly connected to the mouth of the user, it is very likely that the condensation directly flows into the user's mouth, which mitigates the efficacy and causes a poor user experience.

Therefore, to solve this technical problem, an outflow stopper 23 is provided on a bottom wall 200d inner surface of the mouthpiece 2; the top wall 200c and the bottom wall of the mouthpiece 2 are described when the mouthpiece 2 is in a normal spraying state. The condensation of the aerosol is stopped by the outflow stopper 23 when flowing towards the aerosol outflow port 202. By providing the outflow stopper 23, most of the condensation remains inside the mouthpiece 2 instead of directly flowing into the user's mouth, specifically referring to FIGS. 8 and 9.

Specifically, the outflow stopper 23 in this embodiment comprises an outflow stopping convex rib protruding from a bottom wall 200d inner surface of the mouthpiece 2; the protruded outflow stopping convex rib may have a better flow stopping effect. The protrusion height of the outflow stopping convex rib ranges from 1 mm˜6 mm. If the protrusion height is too low, the flow stopping effect will be poor; if the protrusion height is too high, an air flow inside the mouthpiece 2 will be disturbed. Therefore, the protrusion height is generally selected to be 3 mm or 4 mm.

The outflow stopper 23 in this embodiment may also be an outflow stopping groove provided on a bottom wall 200d inner surface of the mouthpiece 2, a structure of which is similar to the diverting groove above; a role played thereby is to partition a smooth inner surface of the mouthpiece 2 such that a flow stopping effect may be implemented to a certain extent; when the outflow stopper 23 is an outflow stopping groove, a depth of the outflow stopping groove ranges from 1 mm˜5 mm; if the depth is too shallow, the flow stopping effect is poor; if the depth is too deep, it will be demanding on the wall thickness of the mouthpiece 2; therefore, the depth of the outflow stopping groove is 2 mm or 3 mm.

In this embodiment, the shape of the aerosol outflow port 202 of the mouthpiece 2 is matched to the shape of the aerosol outflow port 202, i.e., a rectangular shape. However, considering that some children have smaller mouths, such a rectangular aerosol outflow port 202 provides a poor comfort. Therefore, the shape of the aerosol outflow port 202 may be appropriately adapted, e.g., set to be oval shown in FIG. 11.

Additionally, this embodiment also makes improvement to the anti-backflow of the mouthpiece. At least one temporary liquid reservoir 21 is provided in the mouthpiece 2 in this embodiment; when the aerosol condensation flows back to the aerosol inflow port 201, at least part of the aerosol condensation flows back inside the temporary liquid reservoir 21.

As mentioned above, some small-sized condensation will be condensed into relatively large-sized condensation; when the nebulizer is shaken or moved to another location, the aerosol condensation in the mouthpiece 2 likely flows back, i.e., likely flowing back inside the aerosol inflow port 201. Further, the aerosol inflow port 201 is fitted to the nebulize unit; if the condensation flows back to the aerosol inflow port 201, it very likely flows to the nebulize unit, thereby being pooled on the nebulize unit. In this embodiment, a temporary liquid reservoir 21 for reducing or mitigating flowback of the condensation to the aerosol inflow port 201 is provided in the mouthpiece 2. With this design, when flowback of the aerosol occurs, at least part of the aerosol will flow back into the temporary liquid reservoir 21, thereby significantly reducing the aerosol flowing back to the aerosol inflow port 201 and further reducing the odds of being pooled on the nebulize unit.

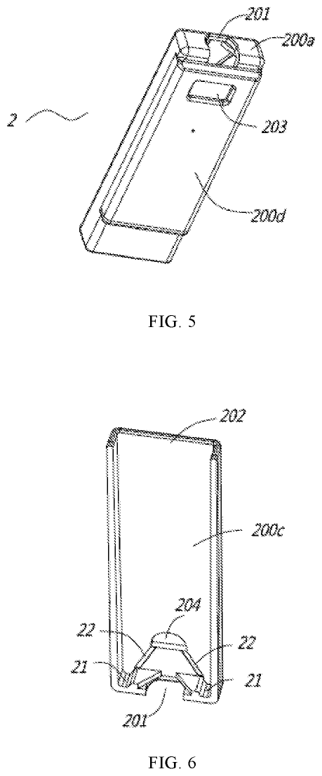

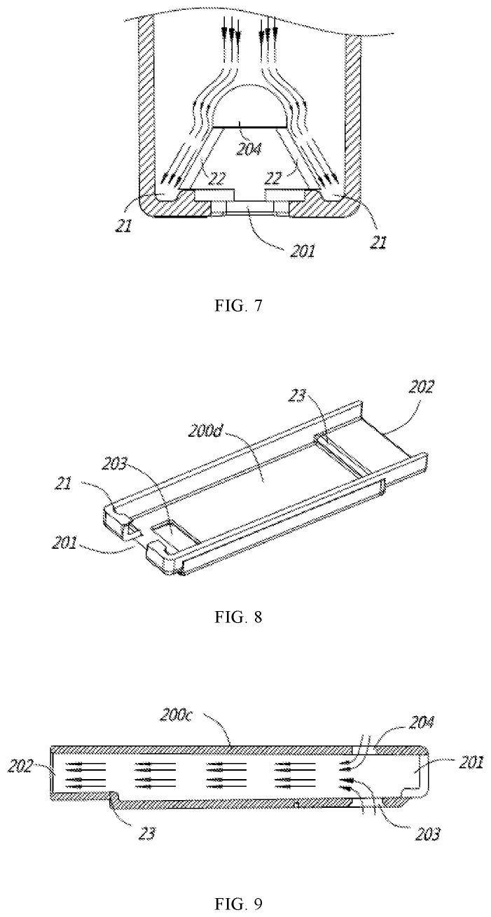

As shown in FIGS. 4 and 5, the mouthpiece 2 in this embodiment is substantially in an elongated cubic shape. The mouthpiece 2 comprises a first end wall 200a and a second end wall 200b which are oppositely arranged; the aerosol inflow port 201 is disposed at the first end wall 200a; the aerosol outflow port 202 is disposed at the second end wall 200b; as further shown in FIGS. 6 and 7, the temporary liquid reservoir 21 and the aerosol inflow port 201 are both disposed at the first end wall 200a, such that the structure of the temporary liquid reservoir 21 may not disrupt the air flow direction inside the mouthpiece 2; if they are disposed on the other inner surface, there may have a hidden risk of disrupting the air flow. Of course, if disruption of the air flow is ignored, the temporary liquid reservoir 21 may be provided on other inner side surface in other embodiments.

Additionally, to better divert the pooled liquid, the aerosol inflow port 201 is disposed at a middle position of the first end wall 200a. The temporary liquid reservoir 21 is provided in two. The two temporary liquid reservoirs 21 are disposed at two different sides of the aerosol inflow port 201, such that when the condensation flows back, it may be diverted to the two sides more uniformly, instead of collectively flowing back into one temporary liquid reservoir 21 thereof, thereby providing a better diverting effect.

Additionally, to further enhance the condensation anti-flowback effect, a stopper member 22 for stopping the condensation from flowing back to the aerosol inflow port 201 is provided on an inner side surface of the mouthpiece 2. By providing the stopper member 22, when the condensation flows back towards the aerosol inflow port 201 from the aerosol outflow port 202, the condensation will be stopped by the stopper member 22, thereby stopping or delaying the condensation from flowing towards the aerosol inflow port 201. The structure and shape of the stopper member 22 are provided in varieties, and this embodiment selects one therefrom, as shown in FIGS. 6 and 7.

In this embodiment, the stopper member 22 comprises a diverting groove disposed on the inner side surface of the mouthpiece 2; one end of the diverting groove extends towards the temporary liquid reservoir 21; the diverting groove plays a role of partitioning the smooth inner surface of the mouthpiece 2, such that when the condensation flows back to the diverting groove, if the amount is small, the condensation is directed to the temporary liquid reservoir 21 along an edge of the diverting groove; if the amount is relatively large, the condensation directly enters inside the diverting groove and then is directed to the temporary liquid reservoir 21.

To further prevent the condensation from flowing back to the nebulize unit, this embodiment further improves the aerosol inflow port 201; in a normal spraying state, a bottom wall 200d is provided at one side of the mouthpiece 2 towards the housing 1, and an opening of the aerosol inflow port is through from an end wall of the mouthpiece 2 to an outer surface of the bottom wall 200d of the mouthpiece 2. Such a design has a purpose that when the condensation flows back to the aerosol inflow port 201, part of the condensation directly flows out downwardly from the bottom wall 200d instead of flowing onto the nebulize unit, such that less pooling is produced on the nebulize unit.

Additionally, in this embodiment, besides providing a main air inlet 203 on the mouthpiece 2, an ancillary air inlet 204 is further provided through an inner side surface of the mouthpiece 2. By providing the ancillary air inlet 204, an air inlet amount increases; further, by providing the ancillary air inlet 204 through the inner side surface, a partition is formed on the inner side surface, which may further play a role of stopping the back-flowing condensation. In this embodiment, as mentioned above, the stopper member 22 comprises two diverting grooves. The two diverting grooves extend to the temporary liquid reservoir 21 from two sides of the ancillary air inlet 204, respectively; in this embodiment, the ancillary air inlet 204 and the two diverting grooves form an isolated area, and the aerosol inflow port 201 is just disposed in the isolated area, while the temporary liquid reservoir 21 is disposed outside of the isolated area. In this way, when the condensation flows back to the first end wall 200a, it is substantially stopped outside the isolated area and substantially does not enter the isolated area.

In an embodiment, the main air inlet 203 of the mouthpiece 2 is disposed at the bottom wall 200d of the mouthpiece 2; because the main air inlet 203 is provided through the bottom wall 200d inner surface of the mouthpiece 2 and the main air inlet 203 in this embodiment is disposed at a relatively middle position; when the condensation flows back, the main air inlet 203 may also stop the condensation from the bottom wall 200d of the mouthpiece 2 and direct the condensation to flow towards the temporary liquid reservoirs 21 at two sides.

Additionally, as mentioned above, in order to increase the air inlet amount, an ancillary air inlet 204 is provided through the inner side surface of the mouthpiece 2 in this embodiment. More specifically, the main air inlet 203 may be disposed at the bottom wall 200d of the mouthpiece 2, while the ancillary air inlet 204 may be disposed at a top wall 200c of the mouthpiece 2. A purpose of providing the ancillary air inlet 204 on the top wall 200c of the mouthpiece 2 is to increase the air inlet amount, which facilitates pushing forward the air flow; meanwhile, because the air inlets are disposed at the top wall 200c and the bottom wall 200d of the mouthpiece 2, the air flow in the mouthpiece 2 is more concentrated in an area between the inner surface of the top wall 200c and the inner surface of the bottom wall 200d, such that the aerosol should be kept away from the top wall 200c and the bottom wall 200d as much as possible, and more aerosol may reach into the user's mouth, instead of being adsorbed to the inner surface of the mouthpiece 2. For details, please refer to the air flow schematic diagram of FIG. 9.

An initial purpose of providing the ancillary air inlet 204 on the top wall 200c of the mouthpiece 2 is to increase the air inlet amount; however, many experiments show that an unexpected effect may be caused by providing the ancillary air inlet 204 there. The diameter of gaseous particles of existing aerosol is substantially between 1 micron and 5 microns; while an actual effective gaseous particle diameter is about 3 microns, because when the gaseous particles have a diameter of about 1 micron, the too small diameter causes the gaseous particles to be exhaled easily when breathing, such that they cannot enter into the body; when the gaseous particles have a diameter of about 4 microns, they generally can only reach the throat; while when the gaseous particles have a diameter of about 5 microns, the larger diameter causes these gaseous particles to substantially only reside in the mouth and unable to be inhaled into the lung. In actual tests, it is found that the gaseous particles with a diameter of about 3 microns is most appropriate and effective, which may be inhaled into the lungs but can be hardly exhaled during breathing.

Further, in this embodiment, after the ancillary air inlet 204 is disposed at the top wall 200c of the mouthpiece 2, many tests find that most of the gaseous particles of the aerosol discharged from the aerosol outflow port 202 have a diameter of about 3 microns. Such kind of aerosol is easily inhaled into the lung, which may significantly enhance the treatment efficacy of the nebulizer.

Additionally, many tests show that when the ancillary air inlet 204 is disposed at a side adjacent to the aerosol inflow port 201. Generally, a maximum distance D1 between the ancillary air inlet 204 and the first end wall 200a does not exceed 4.5 cm; when the distance exceeds 4.5 cm, the gaseous particles with a diameter of around 3 microns among the gaseous particles produced at the aerosol outflow port 202 decrease significantly.

Meanwhile, a minimum distance D2 between the ancillary air inlet 204 and the first end wall 200a may be no less than 1.2 cm. If the minimum distance is too short, the ancillary air inlet 204 of the mouthpiece 2 top wall 200c will be blocked by the housing 1 in a normal spraying state, such that it cannot play a function of assisting air inlet.

Additionally, the ancillary air inlet 204 in this embodiment has a D shape. Tests show that with the ancillary air inlet 204 of this shape, the air flow inside the mouthpiece 2 is more stable, and more gaseous particles with a diameter of around 3 microns will be produced. Of course, in other embodiments, the ancillary air inlet may be of a rectangular, oval or triangular shape.

### Embodiment II

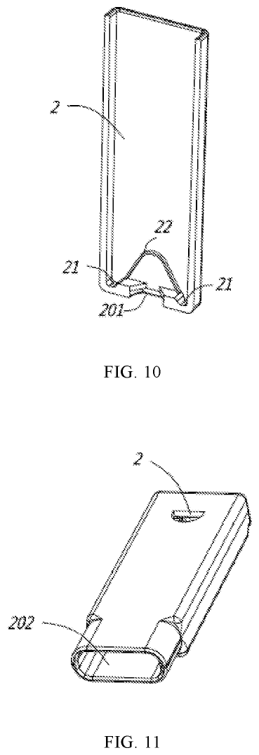

As shown in FIG. 10, the stopper member 22 in this embodiment is not a diverting groove, but a diverting convex rib. When the condensation flows back reversely, it is blocked by the diverting convex rib, thereby implementing an effect of stopping the condensation from flowing back to the aerosol inflow port 201.

Additionally, in this embodiment, an ancillary air inlet 204 is not disposed on the mouthpiece 2; in this embodiment, the entire diverting convex rib encloses an isolated area. In other words, for the isolated area, isolation may be implemented only with the stopper member 22. For example, in this embodiment, the ancillary air inlet 204 and the stopper member 22 jointly form an isolated area, as presented in Embodiment 1.

### Embodiment III

As shown in FIGS. 1-3, a portable nebulizer is provided in this embodiment. The portable nebulizer comprises a housing 1, a nebulize unit provided to the housing 1, and a mouthpiece 2 movably mounted on the housing 1; in this embodiment, the mouthpiece 2 is detachably mounted to the housing 1; specifically, they may be fitted via magnetic attachment. The mouthpiece 2 in this embodiment adopts the mouthpiece 2 shown in Embodiment 1 or Embodiment 2 or an equivalence thereto; in the normal spraying state, the aerosol inflow port 201 is fitted to the nebulize unit.

The nebulizer in this embodiment is portable such that it is very easy to carry. In an embodiment, a receiving cavity 101 is provided on the housing 1, such that when the nebulizer is in a non-spraying state, the mouthpiece 2 is received in the receiving cavity 101, and when the nebulizer is in a spraying state, the mouthpiece 2 is opened and has a working angle a relative to the housing 1. In other words, in the non-spraying state, the mouthpiece 2 may be received, such that the size of the entire nebulizer does not increase, while in use, the mouthpiece 2 is opened to operate, such that it is very convenient to use.

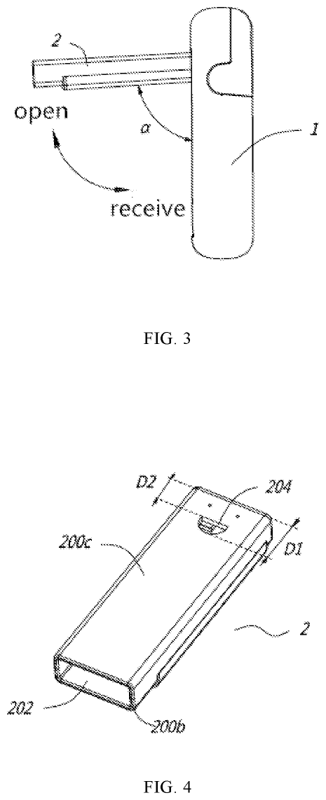

The use angle of the nebulizer in this embodiment is relatively free. Specifically, in the received state, the working angle α ranges from 70° to 95°, for example 85°. In such a working angle α, it is user-friendly. The details are shown in FIG. 3.

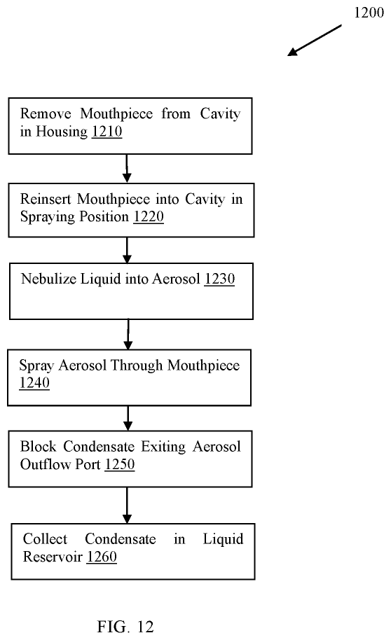

FIG. 12 is a flowchart illustrating a method 1200 of operating a nebulizer according to any of the embodiments disclosed herein. In block 1210, a user removes the mouthpiece from the cavity; and reinserts, at block 1220, the mouthpiece into the cavity in a spraying position; the nebulizer unit then nebulizes the liquid at into an aerosol at block 1230 and sprays the aerosol through the mouthpiece (block 1240). The outflow stopper then blocks condensate exiting the outflow port (block 1250) and a first liquid reservoir collects condensate. The method 1200 then ends. Note that the blocks in the method 1200 can be omitted and/or performed in an order other than described (e.g., simultaneously, block 1260 before block 1250, etc.).

The following examples describe various embodiments of methods and apparatuses (e.g., machines, devices, or other apparatus) discussed herein.

1. A nebulizer mouthpiece, comprising:

an aerosol inflow port,

an aerosol outflow port;

wherein an aerosol passage is formed between the aerosol inflow port and the aerosol outflow port, such that in a spraying state, the aerosol enters the aerosol passage from the aerosol inflow port and is discharged from the aerosol outflow port,

an outflow stopper is provided on an inner surface of the mouthpiece, and condensate from the aerosol is stopped by the outflow stopper when flowing towards the aerosol outflow port;

a first liquid reservoir adjacent the aerosol inflow port; and

a first diverting groove provided on the inner surface configured to divert backflow of the condensate into the reservoir.

2. The nebulizer mouthpiece according to example 1, wherein the outflow stopper comprises an outflow stopping convex rib protruding from the inner surface of the mouthpiece.

3. The nebulizer mouthpiece according to one of the preceding examples, wherein the outflow stopper comprises an outflow stopping groove provided on the inner surface of the mouthpiece.

4. The nebulizer mouthpiece according to one of the preceding examples, wherein the outflow stopper extends from a second inner surface of the mouthpiece to a third inner surface of the mouthpiece, the second and third inner surfaces each form an angle to the first inner surface.

5. The nebulizer mouthpiece according to one of the preceding examples, wherein an outer shape of the aerosol outflow port is of a rectangular, round, or oval shape.

6. The nebulizer mouthpiece according to one of the preceding examples, further comprising a second liquid reservoir and a second diverting groove, the first reservoir disposed on a first side of the aerosol inflow port and the second reservoir disposed on a second side of the aerosol inflow port, the diverting grooves configured to divert condensate into respective first and second reservoirs.

7. The nebulizer mouthpiece according to one of the preceding examples, further comprising an air inlet, a second liquid reservoir and a second diverting groove, the first reservoir disposed on a first side of the aerosol inflow port and the second reservoir disposed on a second side of the aerosol inflow port, the diverting grooves configured to divert condensate into respective first and second reservoirs, the air inlet disposed at first ends of the diverting grooves.

8. A nebulizer, comprising:
a housing,
a nebulize unit within the housing, and
a mouthpiece removably mounted to a cavity in the housing and flush with the housing in a non-spraying state, wherein the mouthpiece is configured to be removed from the cavity in the non-spraying state and reinserted into the cavity in a spraying state at an angle to the housing; the mouthpiece comprising
an aerosol inflow port,
an aerosol outflow port;
wherein an aerosol passage is formed between the aerosol inflow port and the aerosol outflow port, such that in a spraying state, the aerosol enters the aerosol passage from the aerosol inflow port and is discharged from the aerosol outflow port,
an outflow stopper is provided on an inner surface of the mouthpiece, and condensate from the aerosol is stopped by the outflow stopper when flowing towards the aerosol outflow port;
a first liquid reservoir adjacent the aerosol inflow port; and
a first diverting groove provided on the inner surface configured to divert backflow of the condensate into the reservoir.

9. The nebulizer according to one of the preceding examples, wherein the outflow stopper comprises an outflow stopping convex rib protruding from the inner surface of the mouthpiece.

10. The nebulizer according to one of the preceding examples, wherein the outflow stopper comprises an outflow stopping groove provided on the inner surface of the mouthpiece.

11. The nebulizer according to one of the preceding examples, wherein the outflow stopper extends from a second inner surface of the mouthpiece to a third inner surface of the mouthpiece, the second and third inner surfaces each form an angle to the first inner surface.

12. The nebulizer according to one of the preceding examples, wherein an outer shape of the aerosol outflow port is of a rectangular, round, or oval shape.

13. The nebulizer according to one of the preceding examples, wherein the mouthpiece further comprises a second liquid reservoir and a second diverting groove, the first reservoir disposed on a first side of the aerosol inflow port and the second reservoir disposed on a second side of the aerosol inflow port, the diverting grooves configured to divert condensate into respective first and second reservoirs.

14. The nebulizer according to one of the preceding examples, wherein the mouthpiece further comprises an air inlet, a second liquid reservoir and a second diverting groove, the first reservoir disposed on a first side of the aerosol inflow port and the second reservoir disposed on a second side of the aerosol inflow port, the diverting grooves configured to divert condensate into respective first and second reservoirs, the air inlet disposed at first ends of the diverting grooves.

15. A method of operating a nebulizer, the nebulizer comprising a housing, a nebulize unit within the housing, a mouthpiece removably mounted to a cavity in the housing and flush with the housing in a non-spraying state, wherein the mouthpiece is configured to be removed from the cavity in the non-spraying state and reinserted into the cavity in a spraying state at an angle to the housing; the mouthpiece comprising
an aerosol inflow port,
an aerosol outflow port;
wherein an aerosol passage is formed between the aerosol inflow port and the aerosol outflow port, such that in a spraying state, the aerosol enters the aerosol passage from the aerosol inflow port and is discharged from the aerosol outflow port,
an outflow stopper is provided on an inner surface of the mouthpiece, and condensate from the aerosol is stopped by the outflow stopper when flowing towards the aerosol outflow port;
a first liquid reservoir adjacent the aerosol inflow port; and
a first diverting groove provided on the inner surface configured to divert backflow of the condensate into the reservoir,
the method comprising:
removing the mouthpiece from the cavity;
reinserting the mouthpiece into the cavity in a spraying position;
nebulizing the liquid with the nebulize unit into an aerosol;
spraying the aerosol through the mouthpiece;
blocking condensate exiting the outflow port with the outflow stopper;
collecting condensate with the first liquid reservoir.

16. The method according to one of the preceding examples, wherein the outflow stopper comprises an outflow stopping convex rib protruding from the inner surface of the mouthpiece.

17. The method according to one of the preceding examples, wherein the outflow stopper comprises an outflow stopping groove provided on the inner surface of the mouthpiece.

18. The method according to one of the preceding examples wherein the outflow stopper extends from a second inner surface of the mouthpiece to a third inner surface of the mouthpiece, the second and third inner surfaces each form an angle to the first inner surface.

19. The method according to one of the preceding examples, wherein the mouthpiece further comprises a second liquid reservoir and a second diverting groove, the first reservoir disposed on a first side of the aerosol inflow port and the second reservoir disposed on a second side of the aerosol inflow port, and the method further comprises diverting condensate into first and second reservoirs the respective first and second diverting grooves.

20. The method according to one of the preceding examples, wherein the mouthpiece further comprises an air inlet, a second liquid reservoir and a second diverting groove, the first reservoir disposed on a first side of the aerosol inflow port and the second reservoir disposed on a second side of the aerosol inflow port, the air inlet disposed at first ends of the diverting grooves, and wherein the method further comprises diverting condensate into first and second reservoirs with the respective diverting grooves.

What have been described above are only embodiments of the present disclosure; however, the protection scope of the present disclosure is not limited thereto. A person skilled in the art should understand that the present disclosure includes, but not limited to the contents described in the drawings and the embodiments. Any modifications without departing from the functions and structural principles of the present disclosure will be included within the scope of the claims.

1. A nebulizer mouthpiece, comprising:
an aerosol inflow port,
an aerosol outflow port;
wherein an aerosol passage is formed between the aerosol inflow port and the aerosol outflow port, such that in a spraying state, the aerosol enters the aerosol passage from the aerosol inflow port and is discharged from the aerosol outflow port,
an outflow stopper is provided on an inner surface of the mouthpiece, and condensate from the aerosol is stopped by the outflow stopper when flowing towards the aerosol outflow port;
a first liquid reservoir adjacent the aerosol inflow port; and
a first diverting groove provided on the inner surface configured to divert backflow of the condensate into the reservoir.

2. The nebulizer mouthpiece according to claim 1, wherein the outflow stopper comprises an outflow stopping convex rib protruding from the inner surface of the mouthpiece.

3. The nebulizer mouthpiece according to claim 1, wherein the outflow stopper comprises an outflow stopping groove provided on the inner surface of the mouthpiece.

4. The nebulizer mouthpiece according to claim 1, wherein the outflow stopper extends from a second inner surface of the mouthpiece to a third inner surface of the mouthpiece, the second and third inner surfaces each form an angle to the first inner surface.

5. The nebulizer mouthpiece according to claim 1, wherein an outer shape of the aerosol outflow port is of a rectangular, round, or oval shape.

6. The nebulizer mouthpiece according to claim 1, further comprising a second liquid reservoir and a second diverting groove, the first reservoir disposed on a first side of the aerosol inflow port and the second reservoir disposed on a second side of the aerosol inflow port, the diverting grooves configured to divert condensate into respective first and second reservoirs.

7. The nebulizer mouthpiece according to claim 1, further comprising an air inlet, a second liquid reservoir and a second diverting groove, the first reservoir disposed on a first side of the aerosol inflow port and the second reservoir disposed on a second side of the aerosol inflow port, the diverting grooves configured to divert condensate into respective first and second reservoirs, the air inlet disposed at first ends of the diverting grooves.

8. A nebulizer, comprising:
a housing,
a nebulize unit within the housing, and
a mouthpiece mounted to a cavity in the housing and flush with the housing in a non-spraying state, wherein the mouthpiece is configured to be positioned in the cavity in a spraying state at an angle to the housing; the mouthpiece comprising
an aerosol inflow port,
an aerosol outflow port;
wherein an aerosol passage is formed between the aerosol inflow port and the aerosol outflow port, such that in a spraying state, the aerosol enters the aerosol passage from the aerosol inflow port and is discharged from the aerosol outflow port,
an outflow stopper is provided on an inner surface of the mouthpiece, and condensate from the aerosol is stopped by the outflow stopper when flowing towards the aerosol outflow port;
a first liquid reservoir adjacent the aerosol inflow port; and
a first diverting groove provided on the inner surface configured to divert backflow of the condensate into the reservoir.

9. The nebulizer according to claim 8, wherein the outflow stopper comprises an outflow stopping convex rib protruding from the inner surface of the mouthpiece.

10. The nebulizer according to claim 8, wherein the outflow stopper comprises an outflow stopping groove provided on the inner surface of the mouthpiece.

11. The nebulizer according to claim 8, wherein the outflow stopper extends from a second inner surface of the mouthpiece to a third inner surface of the mouthpiece, the second and third inner surfaces each form an angle to the first inner surface.

12. The nebulizer according to claim 8, wherein an outer shape of the aerosol outflow port is of a rectangular, round, or oval shape.

13. The nebulizer according to claim 8, wherein the mouthpiece further comprises a second liquid reservoir and a second diverting groove, the first reservoir disposed on a first side of the aerosol inflow port and the second reservoir disposed on a second side of the aerosol inflow port, the diverting grooves configured to divert condensate into respective first and second reservoirs.

14. The nebulizer according to claim 8, wherein the mouthpiece further comprises an air inlet, a second liquid reservoir and a second diverting groove, the first reservoir disposed on a first side of the aerosol inflow port and the second reservoir disposed on a second side of the aerosol inflow port, the diverting grooves configured to divert condensate into respective first and second reservoirs, the air inlet disposed at first ends of the diverting grooves.

15. A method of operating a nebulizer, the nebulizer comprising a housing, a nebulize unit within the housing, a mouthpiece mounted to a cavity in the housing and flush with the housing in a non-spraying state, wherein the mouthpiece is configured to be positioned in the cavity in a spraying state at an angle to the housing; the mouthpiece comprising
an aerosol inflow port,
an aerosol outflow port;
wherein an aerosol passage is formed between the aerosol inflow port and the aerosol outflow port, such that in a spraying state, the aerosol enters the aerosol passage from the aerosol inflow port and is discharged from the aerosol outflow port,
an outflow stopper is provided on an inner surface of the mouthpiece, and condensate from the aerosol is stopped by the outflow stopper when flowing towards the aerosol outflow port;
a first liquid reservoir adjacent the aerosol inflow port; and
a first diverting groove provided on the inner surface configured to divert backflow of the condensate into the reservoir,
the method comprising:
positioning the mouthpiece into the cavity in a spraying position;
nebulizing the liquid with the nebulize unit into an aerosol;
spraying the aerosol through the mouthpiece;
blocking condensate exiting the outflow port with the outflow stopper;
collecting condensate with the first liquid reservoir.

16. The method according to claim 15, wherein the outflow stopper comprises an outflow stopping convex rib protruding from the inner surface of the mouthpiece.

17. The method according to claim 15, wherein the outflow stopper comprises an outflow stopping groove provided on the inner surface of the mouthpiece.

18. The method according to claim 15, wherein the outflow stopper extends from a second inner surface of the mouthpiece to a third inner surface of the mouthpiece, the second and third inner surfaces each form an angle to the first inner surface.

19. The method according to claim 15, wherein the mouthpiece further comprises a second liquid reservoir and a second diverting groove, the first reservoir disposed on a first side of the aerosol inflow port and the second reservoir disposed on a second side of the aerosol inflow port, and the method further comprises diverting condensate into first and second reservoirs the respective first and second diverting grooves.

20. The method according to claim 15, wherein the mouthpiece further comprises an air inlet, a second liquid reservoir and a second diverting groove, the first reservoir disposed on a first side of the aerosol inflow port and the second reservoir disposed on a second side of the aerosol inflow port, the air inlet disposed at first ends of the diverting grooves, and wherein the method further comprises diverting condensate into first and second reservoirs with the respective diverting grooves.
